(19) United States
(12) Patent Application Publication
(10) Pub. No.: US 2024/0306612 A1
(43) Pub. Date: Sep. 19, 2024

(54) A SYSTEM AND METHOD FOR MONITORING THE EFFECT OF A HERPESVIRUS-BASED VACCINE IN AN ANIMAL POPULATION

(71) Applicant: INTERVET INC., Madison, NJ (US)

(72) Inventors: Yun-Ting WANG, Madison, NJ (US); Linnea Jean NEWMAN, Madison, NJ (US); Taylor Marcelo CORREA BARBOSA, Madison, NJ (US); Hendrik Cornelius KOOPMAN, Boxmeer (NL)

(21) Appl. No.: 18/570,077

(22) PCT Filed: Jun. 16, 2022
(86) PCT No.: PCT/US2022/033779

§ 371 Date: Dec. 14, 2023

Related U.S. Application Data
(60) Provisional application No. 63/211,640, filed on Jun. 17, 2021.

(51) Int. Cl. A01K 45/00; C12Q 1/6809; C12Q 1/70; G16H 50/30

(52) U.S. Cl. CPC A01K 45/00; C12Q 1/6809; C12Q 1/70; G16H 50/30

(57) ABSTRACT

The presently disclosed subject matter aims to a system and method directed to monitor the effect of a herpesvirus-based vaccine in an animal population. The system and method include a processing circuitry configured to: obtain one or more tissue samples of one or more respective animals of the animal population; sequence each of the tissue samples; calculate a score associated with the animal population based on the sequence of the tissue samples; compare the score to a benchmark determined from a dataset containing data associated with the effect of the herpesvirus-based vaccine in a plurality of animal populations; and, execute an action in response to the comparison to the benchmark.

### TECHNICAL FIELD

The present invention relates to the field of systems and methods for monitoring the effect of a vaccine in an animal population.

### BACKGROUND

Domesticated animals are animals that have been selectively bred and genetically adapted over generations to live alongside humans. Animal domestication falls into three main groupings: domestication for companionship (e.g., dogs and cats), working or draft animals (e.g., horses, donkeys, camels), and animals farmed for food (e.g., sheep, cows, pigs, poultry, etc.).

Much like other animals, the well-being of domesticated animals is continuously threatened by infectious agents or pathogens. For example, in the commercial poultry industry, pathogen-produced diseases such as the Newcastle disease (ND), the infectious bursal disease (IBD), the infectious laryngotracheitis (ILT) disease, the Avian Influenza (AI) disease, and the Marek's disease (MD) pose a constant threat to the welfare of poultry flocks.

To protect poultry flocks from diseases, poultry farmers rely on, for example, vaccination and biosecurity. Vaccination programs and vaccine application methods vary based on multiple factors, such as types of production (egg-producing birds, breeder birds, or meat-type birds), types of vaccine (live (e.g., live, live attenuated, killed. DNA-based, or recombinant), types of a pathogen, disease prevalence, local preference, and cost. Regardless of what vaccine is used or how it is applied, the ultimate goal of vaccination is to achieve immunologic protection against a specific disease.

One efficient way to do so is by using recombinant vaccines. Recombinant vaccines are vaccines produced through recombinant DNA technology involving the insertion of a DNA encoding an antigen (such as a pathogen surface protein, e.g., bacterial surface protein, viral surface protein, etc.) so as to stimulate an immune response.

Due to its intrinsic properties of being capable of incorporating large amounts of foreign genetic material, undergoing replication-competent persistent infections in the host, and shifting between an active phase and a latent phase (in which it creates long term immunity), the herpesvirus is one preferred candidate in recombinant vaccines to serve as a carrier of target viral genes of pathogens possessing a threat to the well-being of animal populations. That said, the attribute of the herpesvirus allowing it to shift between a latent phase and an active phase, along with its high variability from animal to animal and from breed to breed, also produces difficulties when trying to monitor or evaluate the effect of a herpesvirus-based vaccine. For example, a poultry flock vaccinated with a herpesvirus-based vaccine can include birds possessing varying levels of herpesvirus loads over time, which creates uncertainty when monitoring or determining the effect of a herpesvirus-based vaccine in a given vaccinated poultry flock.

Thus, there is a need in the art for a new system and method for monitoring the effect of a herpes-based vaccine in an animal population.

### GENERAL DESCRIPTION

In accordance with a first aspect of the presently disclosed subject matter, there is provided a method for monitoring the effect of a herpesvirus-based vaccine in an animal population comprising: obtaining one or more tissue samples of one or more respective animals of the animal population: sequencing each of the tissue samples; calculating a score associated with the animal population based on the sequencing of the tissue samples; comparing the score to a benchmark determined from a dataset containing data associated with the effect of the herpesvirus-based vaccine in a plurality of animal populations; and, executing an action in response to the comparison to the benchmark.

In one embodiment of the presently disclosed subject matter and/or embodiments thereof, herpesvirus-based vaccine involves the use of a turkey Herpesvirus (HVT).

In one embodiment of the presently disclosed subject matter and/or embodiments thereof, the animal population is a flock of poultry.

In one embodiment of the presently disclosed subject matter and/or embodiments thereof, the flock of poultry is a flock of chickens.

In one embodiment of the presently disclosed subject matter and/or embodiments thereof, following the performing step, each of the tissue samples receives an individual score associated with the level of the herpesvirus-based vaccine within the respective tissue sample.

In one embodiment of the presently disclosed subject matter and/or embodiments thereof, the score associated with the animal population is a weighted arithmetic mean of the individual scores associated with the tissue samples.

In one embodiment of the presently disclosed subject matter and/or embodiments thereof, the one or more tissue samples are samples obtained from one or more organs of the one or more respective animals, the one or more organs consists of. a feather pulp, a spleen, and a bursa of Fabricius.

In one embodiment of the presently disclosed subject matter and/or embodiments thereof, the action involves sending a notification indicating that the score is below the threshold.

In one embodiment of the presently disclosed subject matter and/or embodiments thereof, the notification is provided to an end user.

In one embodiment of the presently disclosed subject matter and/or embodiments thereof, the action involves providing the end user with at least one potential action aimed to improve the score.

In one embodiment of the presently disclosed subject matter and/or embodiments thereof, the one or more tissue samples are imprinted on a designated card.

In one embodiment of the presently disclosed subject matter and/or embodiments thereof, the dataset is continuously updated.

In one embodiment of the presently disclosed subject matter and/or embodiments thereof, the dataset is updated at predetermined time periods.

In one embodiment of the presently disclosed subject matter and/or embodiments thereof, the one or more tissue samples are obtained from the one or more respective animals at specific age range.

In one embodiment of the presently disclosed subject matter and/or embodiments thereof, the specific age range is between day 21 and day 25.

In accordance with a second aspect of the presently disclosed subject matter, there is provided a system for monitoring the effect of a herpesvirus-based vaccine in an animal population comprising a processing circuitry configured to: obtain or more tissue samples of one or more respective animals of the animal population; sequence each of the tissue samples; calculate a score associated with the animal population based on the sequence of the tissue samples; compare the score to a benchmark determined from a dataset containing data associated with the effect of the herpesvirus-based vaccine in a plurality of animal populations; and, execute an action in response to the comparison to the benchmark.

In one embodiment of the presently disclosed subject matter and/or embodiments thereof, the herpesvirus-based vaccine involves the use of a turkey Herpesvirus (HVT).

In one embodiment of the presently disclosed subject matter and/or embodiments thereof, the animal population is a flock of poultry.

In one embodiment of the presently disclosed subject matter and/or embodiments thereof, the flock of poultry is a flock of chickens.

In one embodiment of the presently disclosed subject matter and/or embodiments thereof, following the performing step, each of the tissue samples receives an individual score associated with the level of the herpesvirus-based vaccine within the respective tissue sample.

In one embodiment of the presently disclosed subject matter and/or embodiments thereof, the score associated with the animal population is a weighted arithmetic mean of the individual scores associated with the tissue samples.

In one embodiment of the presently disclosed subject matter and/or embodiments thereof, the one or more tissue samples are samples obtained from one or more organs of the one or more respective animals, the one or more organs consists of: a feather pulp, a spleen, and a bursa of Fabricius.

In one embodiment of the presently disclosed subject matter and/or embodiments thereof, the action involves sending a notification indicating that the score is below the threshold.

In one embodiment of the presently disclosed subject matter and/or embodiments thereof, the notification is provided to an end user.

In one embodiment of the presently disclosed subject matter and/or embodiments thereof, the action involves providing the end user with at least one potential action aimed to improve the score.

In one embodiment of the presently disclosed subject matter and/or embodiments thereof, the one or more tissue samples are imprinted on a designated card.

In one embodiment of the presently disclosed subject matter and/or embodiments thereof, the dataset is continuously updated.

In one embodiment of the presently disclosed subject matter and/or embodiments thereof, the dataset is updated at predetermined time periods.

In one embodiment of the presently disclosed subject matter and/or embodiments thereof, the one or more tissue samples are obtained from the one or more respective animals at specific age range.

In one embodiment of the presently disclosed subject matter and/or embodiments thereof, the specific age range is between day 21 and day 25.

In accordance with a third aspect of the presently disclosed subject matter, there is provided a non-transitory computer readable storage medium having computer readable program code embodied therewith, the computer readable program code, executable by at least one processor to perform a method for monitoring the effect of a herpesvirus-based vaccine in an animal population, the monitoring of the effect of a herpesvirus-based vaccine comprising one or more components, the method comprising: obtaining one or more tissue samples of one or more respective animals of the animal population: sequencing each of the tissue samples; calculating a score associated with the animal population based on the sequencing of the tissue samples; comparing the score to a benchmark determined from a dataset containing data associated with the effect of the herpesvirus-based vaccine in a plurality of animal populations; and, executing an action in response to the comparison to the benchmark.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a schematic illustration of an operation of a system for monitoring the effect of a herpesvirus-based vaccine in an animal population, in accordance with the presently disclosed subject matter;

FIG. 2 is a block diagram schematically illustrating one example of a system for monitoring the effect of a herpesvirus-based vaccine in an animal population, in accordance with the presently disclosed subject matter.

FIG. 3 is a flowchart illustrating one example of a sequence of operations carried out by a system for monitoring the effect of a herpesvirus-based vaccine in an animal population, in accordance with the presently disclosed subject matter;

FIGS. 4A-4D are graphs illustrating one example of the ratio of positive samples percentage in different breeds, at different ages, in accordance with the presently disclosed subject matter

FIG. 5 is a graph illustrating one example of different trends in different breeds of the same animal type, in accordance with the presently disclosed subject matter;

FIGS. 6A-6B are schematic illustrations of an operation of a summary score system, in accordance with the presently disclosed subject matter;

FIGS. 7A-7C are dashboards illustrations of examples of the state of a score of an animal population, compared to a threshold or benchmark, in accordance with the presently disclosed subject matter; and,

FIG. 8 is a dot graph illustrating one example of the state of vaccination quality in an animal population, compared to a threshold or benchmark, over time, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “sequencing”, “calculating”, “comparing”, “executing”, “receiving” or the like, include action and/or processes of a computer that manipulate and/or transform data into other data, said data represented as physical quantities, e.g., such as electronic quantities, and/or said data representing the physical objects. The terms “computer”, “processor”, “processing resource”, “processing circuitry”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

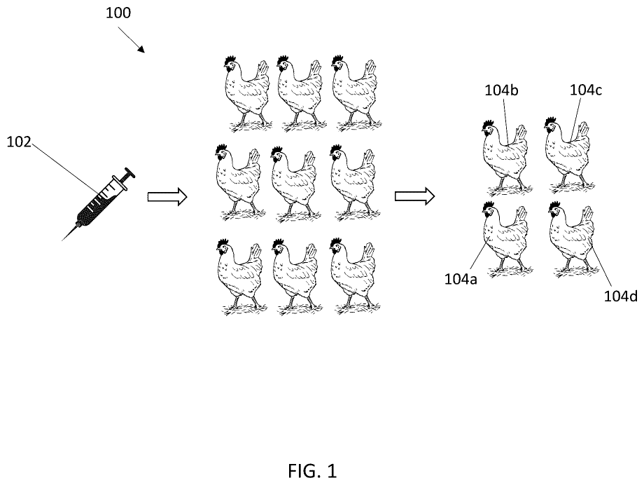

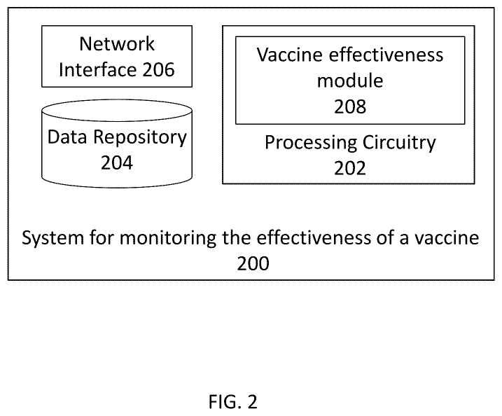

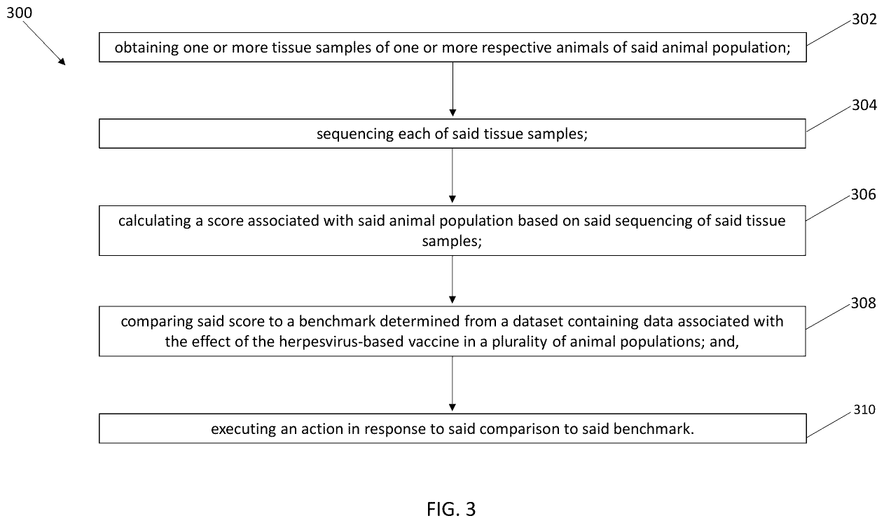

In embodiments of the presently disclosed subject matter, fewer, more and/or different stages than those shown in FIG. 3 may be executed. In embodiments of the presently disclosed subject matter one or more stages illustrated in FIG. 3 may be executed in a different order and/or one or more groups of stages may be executed simultaneously. FIGS. 1 and 2 illustrate a general schematic of the system architecture in accordance with an embodiment of the presently disclosed subject matter. Each module in FIG. 2 can be made up of any combination of software, hardware and/or firmware that performs the functions as defined and explained herein. The modules in FIG. 2 may be centralized in one location or dispersed over more than one location. In other embodiments of the presently disclosed subject matter, the system may comprise fewer, more, and/or different modules than those shown in FIG. 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Any reference in the specification to the term “effect” throughout the text can be directed, for example, to refer to effectiveness, and more specifically, to vaccine take.

By way of introduction, the presently disclosed subject matter provides a way of monitoring a vaccine program in a population of animals, for example, in a population of animals that are difficult to vaccinate, where the monitoring of the success, uptake, and/or effectiveness of the vaccination is considered to be challenging.

Bearing this in mind, attention is drawn to FIG. 1, showing a schematic illustration of an operation of a system for monitoring the effect of a herpesvirus-based vaccine in an animal population (also interchangeably referred to herein as “system”), in accordance with the presently disclosed subject matter.

As shown in the schematic illustration, an animal population 100, including, for example, a flock of poultry, is being vaccinated using a herpesvirus-based vaccine 102. The herpesvirus-based vaccine 102 can be, for example, a recombinant vaccine involving the use of a Herpesvirus, for example, turkey Herpesvirus (HVT), serving as a carrier of one or more target viral genes of pathogens possessing a threat to the well-being of animal population 100. The one or more target viral genes may include, for example, a fusion (F) gene of the Newcastle disease virus (NDV), a VP2 gene of the Infectious Bursal Disease virus (IBDV), glycoprotein genes of the infectious laryngotracheitis virus (ILTV), etc.

The administration of the herpesvirus-based vaccine 102 can be made, for example, via subcutaneous injection at day one to day five of the chick age, and in a specific example at day one of the chick age, or via embryo injection (in ovo) at day 10 to day 30 of incubation, and in a specific example at day 17 to day 19 of incubation, using, for example, an in-ovo injection machine that forms a tiny hole on the blunt end of the embryonated egg, and delivers, through the tiny hole formed, the herpesvirus-based vaccine to the embryo body or the amniotic fluid.

It is to be noted that the administration of the vaccine 102 can also be done by other methods and techniques known in the art, and at different ages of the chick or chicks. The other methods and techniques can include, for example, eye drop/nasal instillation, spray vaccination, vaccination by dosing pump, drinking water vaccination, feed vaccination, wing web prick method, beak dipping method, and the like.

After the inoculation of animal population 100 with the herpesvirus-based vaccine 102, and once the herpesvirus-based vaccine 102 reaches its replication peak, a group of animals of animal population 100 (represented by reference signs 104a-104d) is selected so as to verify and monitor the effect of the herpesvirus-based vaccine 102 in animal population 100. The replication peak can vary depending on, for example, the injection route, types of birds, types of rHVT, etc. The selection of the group of animals of animal population 100 can be made either randomly or based on various considerations, e.g., their characteristics, their status within the population, and the like.

By way of example, a flock of chickens 100, including approximately 50 chickens, is vaccinated against the Newcastle disease (ND) using a recombinant turkey Herpesvirus (rHVT) vaccine 102. The rHVT vaccine 102, which contains a fusion (F) gene of the Newcastle disease virus (NDV), is injected to the flock of chickens 100 via embryo injection (in ovo) at day 17 to day 19 of incubation. Two to three weeks after the inoculation, a group of twenty chickens is randomly selected so as to monitor and determine the effect of the recombinant turkey Herpesvirus (rHVT) vaccine 102 against the Newcastle disease (ND) in the flock of chickens 100.

Attention is now drawn to a further description of the components of the system for monitoring the effect of a vaccine 200.

FIG. 2 is a block diagram schematically illustrating one example of the system for monitoring the effect of a vaccine 200, in accordance with the presently disclosed subject matter.

In accordance with the presently disclosed subject matter, the system for monitoring the effect of a vaccine 200 (also interchangeably referred to herein as “system 200”) can comprise a network interface 206. The network interface 206 (e.g., a network card, a Wi-Fi client, a Li-Fi client, 3G/4G client, or any other component), enables system 200 to communicate over a network with external systems and handles inbound and outbound communications from such systems. For example, the system for monitoring the effect of a vaccine 200 can receive, through network interface 206, data from an external benchmarking system that can provide it with a dataset associated with the effect of a herpesvirus-based vaccine in a plurality of animal populations.

System 200 can further comprise or be otherwise associated with a data repository 204 (e.g., a database, a storage system, a memory including Read Only Memory—ROM, Random Access Memory—RAM, or any other type of memory, etc.) configured to store data. Some examples of data that can be stored in the data repository 204 include:
The number of tissue samples collected from an animal population 100;
The individual scores associated with the tissue samples collected from the animal population 100;
The score associated with the animal population 100, calculated based on the individual scores of the tissue samples collected from the animal population 100;
The potential actions provided to an end user in response to the score associated with his animal population 100 being below a threshold or benchmark;
Definitions of various notifications that can be provided by the system 200; and
Information on one or more organs from which all or some of the tissue samples were obtained.

Data repository 204 can be further configured to enable retrieval and/or update and/or deletion of the stored data. It is to be noted that in some cases, data repository 204 can be distributed, while the system 200 has access to the information stored thereon, e.g., via a wired or wireless network to which system 200 is able to connect (utilizing its network interface 206).

System 200 further comprises processing circuitry 202. Processing circuitry 202 can be one or more processing units (e.g., central processing units), microprocessors, microcontrollers (e.g., microcontroller units (MCUs)) or any other computing devices or modules, including multiple and/or parallel and/or distributed processing units, which are adapted to independently or cooperatively process data for controlling relevant system 200 resources and for enabling operations related to system's 200 resources.

The processing circuitry 202 comprises a vaccine effect determination module 208, configured to perform an effect analysis process, as further detailed herein, inter alia with reference to FIG. 3.

Tuming to FIG. 3 there is shown a flowchart illustrating one example of a sequence of operations carried out for the system for monitoring the effect of a vaccine 200, in accordance with the presently disclosed subject matter.

Accordingly, the system for monitoring the effect of a vaccine 200 (also interchangeably referred to hereafter as “system 200”) can be configured to perform a monitoring process 300, e.g., using vaccine effect module 208.

For this purpose, in accordance with and following the description above with reference to FIG. 1, system 200 obtains one or more tissue samples of the group of animals selected from the animal population 100 (block 302). The one or more tissue samples, which can be obtained from different organs of the selected animals, such as their feather pulp, spleen, bursa of Fabricius, and the like, can then be processed, for example, by being imprinted on a designated card directed to denature proteins and protect nucleic acids in the samples. In one example, spleen samples are obtained from each chicken of the group of the selected chickens, and each sample is later imprinted on a Whatman® FTA® card.

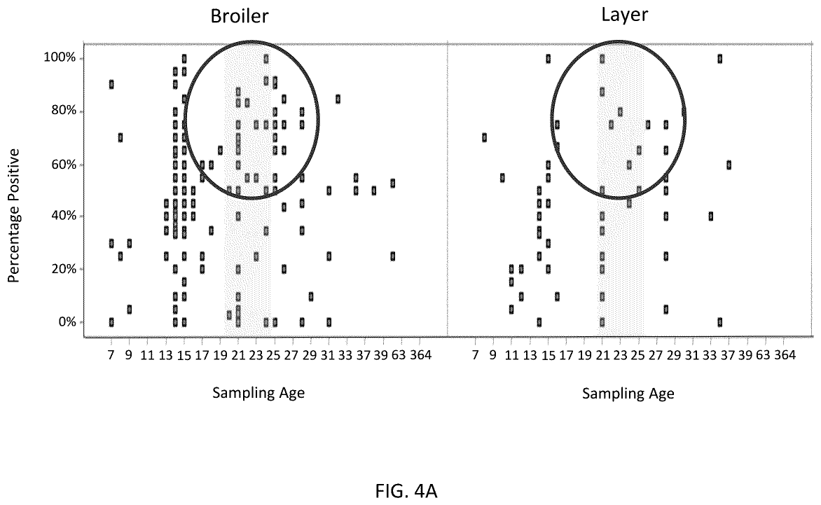

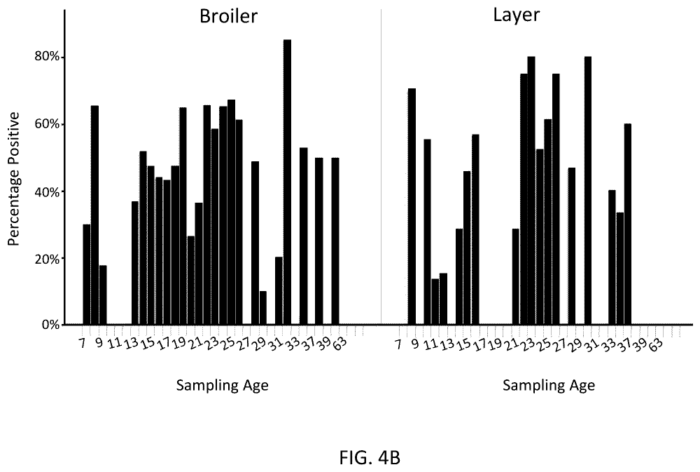

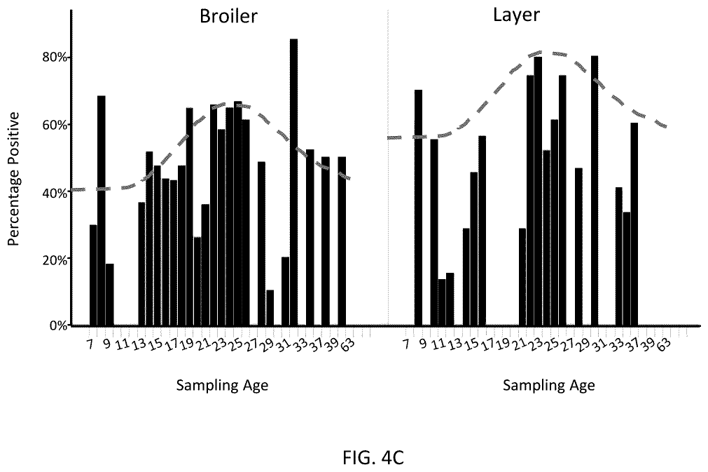

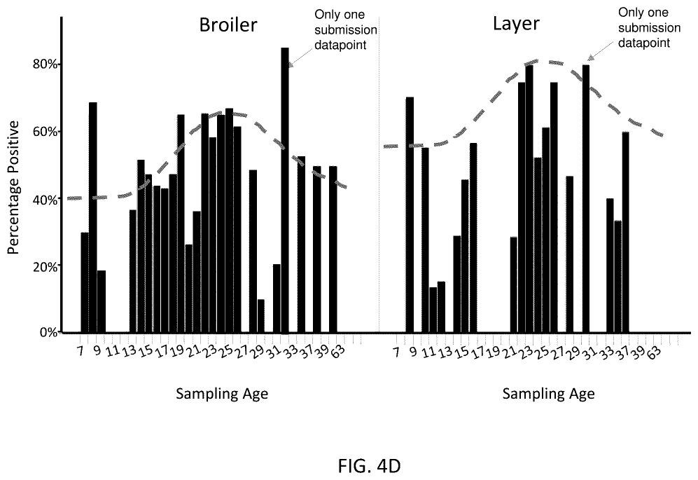

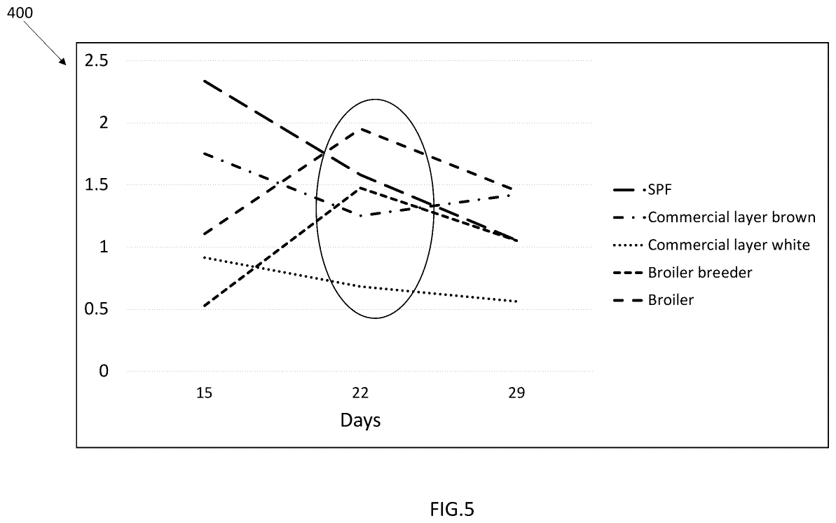

In some cases, the one or more tissue samples are obtained from the group of animals selected at a specific age range, for example, between day 10 and day 30 from birth, and in a more specific example, between day 21 and day 25 from birth. Due to the variability of optimum sampling age with vaccines, and in a particular example, of HVT-based vaccines, identifying the ideal age (or age range) to obtain a tissue sample from the group of animals selected enables system 200 to improve its efficiency and effectiveness. The specific age range can be determined, for example, by analyzing data associated with a plurality of animals within a breed, at different ages, in order to detect a trend within the samples of the plurality of animals. The trend can be detected, for example, by calculating an average positive percentage of samples through different ages and identifying the age range in which the positive percentage peak is considered to be the highest. It is to be noted that the trend within the samples of the plurality of animals may vary with, for example, flock, geography (e.g., animals from different regions, continents, habitats, side of the world, etc.), type of bird (e.g., chickens vs. turkeys, chickens vs. ducks, etc.), breed (e.g., Brahma vs. Buckeye, Chantecler vs. Brahma, etc.), and the like. FIGS. 4A-4D demonstrates the positive percentage of broiler and layer breeds through different ages. As shown in all figures, the age range in which the positive percentage peak is considered to be the highest is between age 21 and age 25, for both breeds. FIG. 5 shows an example of a graph 400 illustrating the trends of 5 different breeds of an animal. The trends were identified based on samples acquired from the feather pulp of a group of animals in each breed. As can be seen in FIG. 5, the trends of the broiler breed and the broiler breeder breed demonstrated their highest peak at day 22. whereas the commercial layer white breed, the commercial layer brown breed, and the SPF breed demonstrated their highest peak at other days. This demonstrates the significant differences in ideal sampling age that can be found within breeds of the same animal, and as such, the different approach that should be implemented even within the same animal type (let alone, in different animal types).

Returning to FIG. 3, once the tissue samples of the group of animals selected from animal population 100 are obtained, system 200 sequences a genetic material (e.g., DNA molecule) extracted from each tissue sample using any sequencing technique known in the art able to perform large-scale sequencing, for example, Next-Generation Sequencing (NGS) (e.g., Roche 454, GS FLX Titanium, Illumina MiSeq, Illumina HiSeq, Illumina Genome Analyzer IIX, Life Technologies SOLID4, Life Technologies Ion Proton. Complete Genomics, Helicos Biosciences Heliscope, Pacific Biosciences SMRT, etc.) (block 304). The sequencing enables amplifying genetic markers associated with the herpesvirus-based vaccine 102 and confirms the presence and quantity of the vaccine in the vaccinated group of animals selected from the animal population 100. In our continuing example, system 200 sequences DNA molecules extracted from each of the tissue samples (in this case spleen tissue samples) obtained from the group of the selected chickens.

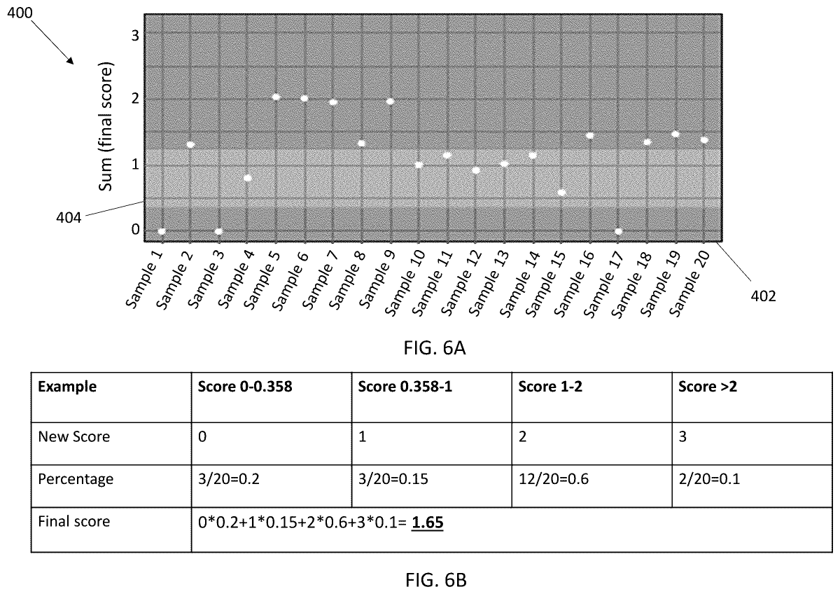

As the sequencing of each tissue sample is completed, system 200 utilizes the tissue samples' sequencing results to calculate a score, which will be associated with the animal population 100 (block 306). The tissue samples' sequencing results can be of either a positive value or a negative value, and can also include a quantification of the virus load (e.g., the number of virus copies in each sample). The score can be determined, for example, by first providing each tissue sample with an individual score associated with the level of the herpesvirus-based vaccine 102 within it, and then calculating a weighted arithmetic mean of the individual scores. Alternatively, the score can be determined by calculating the percentage of positive samples of the tissue samples obtained, or by using a summary score system (best illustrated in FIGS. 6A-6B).

It is to be noted that the summary score system can be part of system 200, or it can be an external system, external thereto, capable of communicating therewith.

As shown in FIG. 6A, and in accordance with our continuing example, following the sequencing of each of the spleen samples of the group of the selected chickens, the summary score system generates, for example, a graph 500 containing an x-axis 502 representing the spleen samples of the selected chickens, a y-axis 504 representing a vaccine test score expressing the number of virus copies found within the spleen samples, normalized to values of between 0 and 3, and twenty dots dispersed thereon, each representing a vaccine test score of a spleen sample obtained from a given chicken.

To determine a score associated with the flock of chickens 100, the summary score system determines a cut-off score, for example, 0.358, score ranges, for example, 0 to 0.358, 0.358 to 1.1 to 2, and above 2, and a list of new scores, for example, 0, 1, 2, and 3, in which each new score is associated with a score range, respectively (see FIG. 6B). The score ranges can be validated, for example, using an external validation dataset. The external validation dataset may include, for example, information regarding the health of each chicken of the group of selected chickens, such that the health of each chicken is directed to be in correlation with its vaccination rate, and consequently, with its corresponding score range.

It is to be noted that the external validation dataset can be part of system 200, or it can be external thereto, capable of communicating therewith.

The summary score system defines the prevalence within each score range by determining the number of dots dispersed therein out of the total number of twenty dots (FIG. 6B, the percentage line), and multiplies it by the new score associated with the corresponding score range (FIG. 6B, the new score line). The results of these calculations are then summarized to a final score, which is the score associated with the flock of chickens 100 (FIG. 6B, the final score line). As shown in FIG. 6B, the score associated with the group of the selected twenty chickens, and by that with the flock of chickens 100, is 1.65.

Once the score associated with animal population 100 is determined, system 200 compares it to a threshold or a benchmark. The threshold or benchmark serves as a reference point representing the industry standard (or the standard of sub groups within the industry) for a particular animal population, e.g., particular bird, particular breed, and the like, such that an end user of system 200 (for example, a farmer) is able to receive information regarding the effect of a vaccine 102 on his animal population 100, compared to the industry standard. Furthermore, the comparison to the threshold or benchmark may further provide the end user with information regarding the effect of the vaccine 102 on his animal population 100, compared to itself and the industry standard, over time. The threshold or benchmark can be determined, for example, by analyzing one or more datasets (that are, for example, continuously updated or updated at predetermined time periods, e.g., every hour, every day, every month, every six months, every year) containing data associated with the effect of the herpesvirus-based vaccine 102 in one or more other animal populations (block 308). The threshold or benchmark can be, for example, the average value of a plurality of scores, each associated with an animal population that received the herpesvirus-based vaccine 102. The one or more other animal populations can vary with, for example, flock, geography (e.g., animals from different regions, continents, habitats, side of the world, etc.), type of bird (e.g., chickens vs. turkeys, chickens vs. ducks, etc.), breed (e.g., Brahma vs. Buckeye, Chantecler vs. Brahma, etc.), and the like. In addition, the respective one or more other animal populations can be, for example, populations of the same animal type, or related animal types that are expected, or known, to have similar results of Herpesvirus-based vaccine effect.

In our continuing example, the threshold or benchmark, which is the average of a plurality of scores associated with a plurality of chicken populations that received the Herpesvirus (rHVT) vaccine 102, is defined to be 1.75. As such, the score associated with the flock of chickens 100 (1.65) is found to be below it.

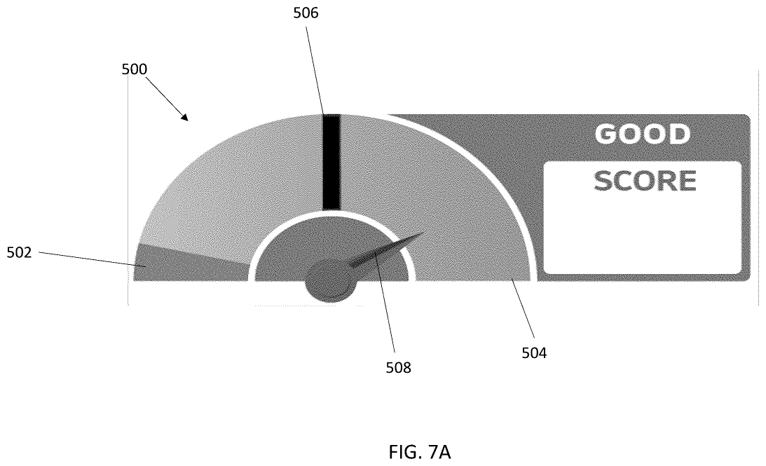

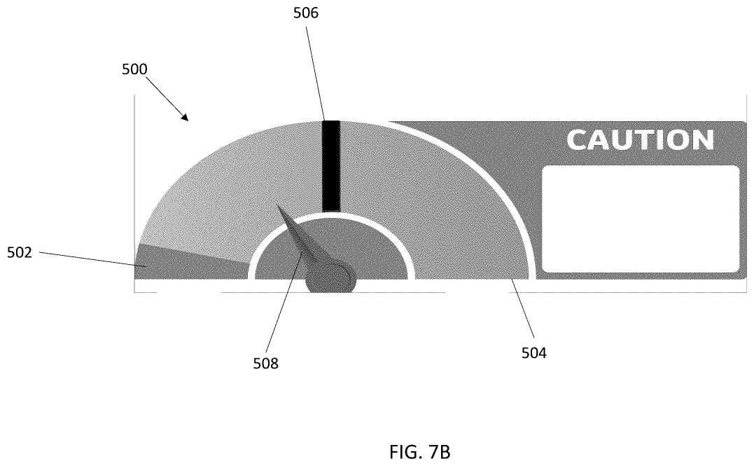

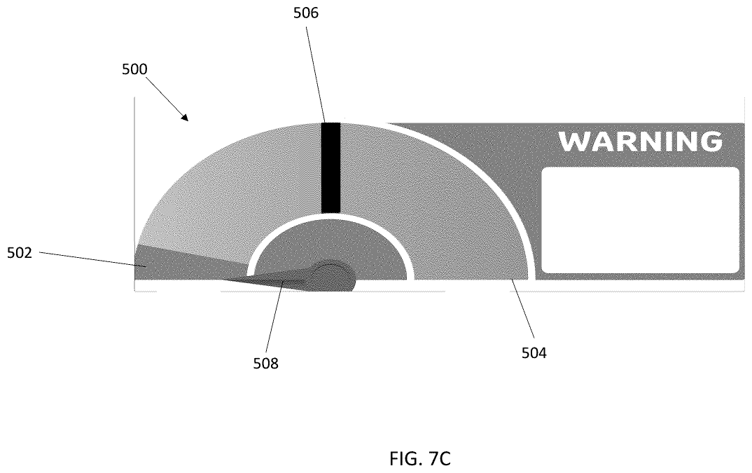

In some cases, as illustrated in FIGS. 7A-7C, the indication of the status of the score associated with animal population 100, compared to the threshold or benchmark, can be presented, for example, on a dashboard 600. As shown in FIGS. 7A-7C, the dashboard 600, which extends from a minimum value range 602 to a maximum value 604, includes a black line 606 representing the threshold (or benchmark) location, and a needle 608 representing the score associated with animal population 100. In cases where the score associated with animal population 100 is above the threshold (or benchmark) value, the needle 608 is to the right of the black line 606, and a “GOOD” notification, along with the score value, is presented to the user (FIG. 7A). In cases where the score associated with animal population 100 is below the threshold (or benchmark) value but above the minimum value range 602, the needle 608 is to the left of the black line 606, and a “CAUTION” notification is presented to the user (FIG. 7B). Finally, in cases where the score associated with animal population 100 falls within the minimum value range 602, a “WARNING” notification is presented to the user (FIG. 7C).

In cases where the score associated with animal population 100 is below the threshold (or benchmark), system 200 executes an action (block 310). The action can be, for example, providing a notification (e.g., to an end user or to an external system) indicating that the score is below the threshold (or benchmark), providing an end user with a recommendation to perform at least one improvement action aimed to improve the score associated with animal population 100, and the like. In our continuing example, as the score of the flock of chickens 100 is below the threshold (or benchmark) (1.65 vs. 1.75), system 200 sends a notification to the end user indicating of the situation, along with a list of improvement actions, including, for example, check hatchery vaccine storage, check hatchery vaccine application, check sample collection, and check vaccine preparation time and temperature. These actions are directed to increase the score associated with animal population 100 (1.65) to be higher, for example at least higher than the threshold (or benchmark) (1.75).

It should be noted that the examples presented above and the values associated with these examples are used for clarification and explanation purposes and are in no way directed to limit the scope of the presently disclosed subject matter.

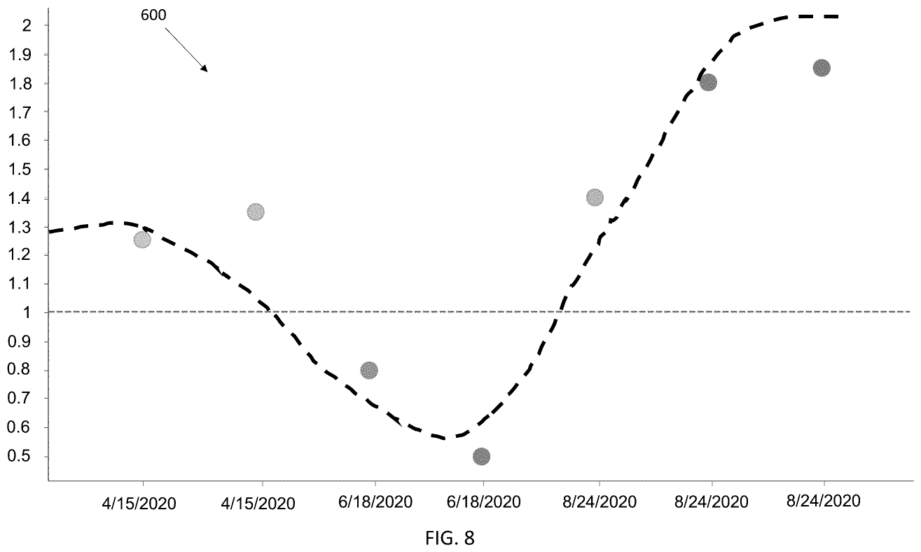

Further to sending notifications or providing potential actions, system 200 can provide an end user with testing information, for example, a graph 700 (as shown in FIG. 8), directed to provide the end user with a presentation of the state of vaccination quality in his animal population 100, compared to the threshold (or benchmark), over time. Altematively or additionally, system 200 can present the testing information to the end user by type of bird (e.g., chicken vs. turkeys), breed (e.g., meat type vs. egg type), and the like, over time.

In some cases, the notifications, potential actions, and testing information are presented to the end user through a mobile application (or other similar software), found in communication with system 200. For example, the mobile application may allow the end user to view the test results associated with his animal population 100, historical testing information associated with his animal population 100, and the comparison of his animal population 100 to the different benchmarks.

In some cases, system 200 further includes a machine learning module that receives additional information from the tissue samples obtained, relating to the success or failure of administering the herpesvirus-based vaccine 102 to the animal population 100. The additional information, which may be collected within a certain time interval from the vaccine administration (e.g., two to three weeks from the vaccine administration), may include, for example, information regarding a possible outbreak in animal population 100 of the disease to which the vaccine 102 was directed, information regarding the production of specific antibodies associated with the specific disease to which the herpesvirus-based vaccine 102 was directed, and the like. Based on the additional information, the machine learning module can determine whether the vaccine administration to animal population 100 was a success or a failure. Furthermore, the machine learning module can be trained, for example, based on the additional information from the tissue samples obtained, to find a correlation between the additional information, the threshold (or benchmark) or the limit values of the dashboard 600, and the actual success or failure of the vaccine, so as to adjust the threshold (or benchmark) or the limit values of the dashboard 600 accordingly. For example, in situations where the benchmark may differ for different bird breeds or types, the machine learning module can adjust the threshold (or benchmark) and the limit values of the dashboard 600 to be in accordance with each bird breed or type.

In other cases, the machine learning module can be trained to utilize additional information received from tissue samples of new breeds or new animal types so as to determine trends within these tissue samples, and from these trends, identify the ideal day range in which tissue samples from the new breeds or the bird types should be obtained.

It is to be noted, with reference to FIG. 3, that some of the blocks can be integrated into a consolidated block or can be broken down to a few blocks and/or other blocks may be added. It is to be further noted that some of the blocks are optional. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A method for monitoring the effect of a herpesvirus-based vaccine in an animal population comprising:
obtaining one or more tissue samples of one or more respective animals of said animal population;
sequencing each of said tissue samples;
calculating a score associated with said animal population based on said sequencing of said tissue samples;
comparing said score to a benchmark determined from a dataset containing data associated with the effect of the herpesvirus-based vaccine in a plurality of animal populations; and,
executing an action in response to said comparison to said benchmark.

2. The method of claim 1, wherein said herpesvirus-based vaccine involves the use of a turkey Herpesvirus (HVT).

3. The method of claim 1, wherein said animal population is a flock of poultry.

4. (canceled)

5. The method of claim 1, wherein, following the performing step, each of said tissue samples receives an individual score associated with the level of said herpesvirus-based vaccine within the respective tissue sample.

6. The method of claim 5, wherein said score associated with said animal population is a weighted arithmetic mean of the individual scores associated with said tissue samples.

7. (canceled)

8. The method of claim 1, wherein said action involves sending a notification to an end user, indicating that said score is below said threshold.

9. (canceled)

10. The method of claim 8, wherein said action further involves providing the end user with at least one potential action aimed to improve said score.

11. (canceled)

12. (canceled)

13. (canceled)

14. The method of claim 1, wherein said one or more tissue samples are obtained from said one or more respective animals at specific age range.

15. The method of claim 14, wherein said specific age range is between day 21 and day 25.

16. A system for monitoring the effect of a herpesvirus-based vaccine in an animal population comprising a processing circuitry configured to:
obtain or more tissue samples of one or more respective animals of said animal population;
sequence each of said tissue samples;
calculate a score associated with said animal population based on said sequence of said tissue samples;
compare said score to a benchmark determined from a dataset containing data associated with the effect of the herpesvirus-based vaccine in a plurality of animal populations; and,
execute an action in response to said comparison to said benchmark.

17. The system of claim 16, wherein said herpesvirus-based vaccine involves the use of a turkey Herpesvirus (HVT).

18. The system of claim 16, wherein said animal population is a flock of poultry.

19. (canceled)

20. The system of claim 16, wherein, following the performing step, each of said tissue samples receives an individual score associated with the level of said herpesvirus-based vaccine within the respective tissue sample.

21. The system of claim 20, wherein said score associated with said animal population is a weighted arithmetic mean of the individual scores associated with said tissue samples.

22. The system of claim 16, wherein said one or more tissue samples are samples obtained from one or more organs of said one or more respective animals, said one or more organs consists of: a feather pulp, a spleen, and a bursa of Fabricius.

23. The system of claim 16, wherein said action involves sending a notification to an end user, indicating that said score is below said threshold.

24. (canceled)

25. The system of claim 23, wherein said action further involves providing the end user with at least one potential action aimed to improve said score.

26. (canceled)

27. (canceled)

28. (canceled)

29. The system of claim 16, wherein said one or more tissue samples are obtained from said one or more respective animals at specific age range.

30. The system of claim 29, wherein said specific age range is between day 21 and day 25.

31. A non-transitory computer readable storage medium having computer readable program code embodied therewith, the computer readable program code, executable by at least one processor to perform a method for monitoring the effect of a herpesvirus-based vaccine in an animal population, the monitoring of the effect of a herpesvirus-based vaccine comprising one or more components, the method comprising:
obtaining one or more tissue samples of one or more respective animals of said animal population;
sequencing each of said tissue samples;
calculating a score associated with said animal population based on said sequencing of said tissue samples;
comparing said score to a benchmark determined from a dataset containing data associated with the effect of the herpesvirus-based vaccine in a plurality of animal populations; and,
executing an action in response to said comparison to said benchmark.
